(19) United States
(12) Patent Application Publication
(10) Pub. No.: US 2018/0014721 A1
(43) Pub. Date: Jan. 18, 2018

(54) SPECULUM RETRACTOR

(71) Applicant: Rultract, Inc., Cleveland, OH (US)

(72) Inventors: Philip M. Rullo, Warren, OH (US); Ryan Tietz, Southington, OH (US); William Koteles, Cleveland, OH (US)

(21) Appl. No.: 15/649,874

(22) Filed: Jul. 14, 2017

Related U.S. Application Data
(60) Provisional application No. 62/362,048, filed on Jul. 14, 2016.

(51) Int. Cl. A61B 1/303; A61B 1/32

(52) U.S. Cl. CPC A61B 1/303; A61B 1/32

(57) ABSTRACT

A medical instrument for spreading tissue includes a main body portion and upper and lower arms coupled to the main body portion. One of the arms is a translatable first arm that translates relative to the main body portion. Upper and lower fingers are coupled to the upper and lower arms, respectively, for engaging and spreading the tissue upon movement of one of the fingers. A translating mechanism adjusts to translate the translatable first arm along the main body portion between a primary position adjacent the other of the arms, referred to as a second arm, and a secondary position spaced from the primary position. An adjustment member engages a lever portion extending from the second arm and causes movement of the lever portion towards and away from the main body portion to adjust an angle between the fingers.

### RELATED APPLICATIONS

This application claims the benefit of U.S. Provisional Application No. 62/362,048 filed Jul. 14, 2016, which is hereby incorporated herein by reference in its entirety.

### FIELD OF INVENTION

The present invention relates generally to a spreading instrument for spreading tissue, and more particularly to a medical instrument for allowing easy, accurate and secure spreading of tissue, particularly by gloved medical personnel.

### BACKGROUND

Spreading instruments, such as retractors or speculums, are often used to spread body tissue, such as to increase the diameter of an orifice, cavity, or incision during medical procedures. Tissue being spread may include an existing body orifice, such as the vagina, mouth, etc., or an incision created in the body, such as an incision of the stomach to obtain access to gastrointestinal tissues. The spreading instrument then enables access into a body, for example, via the space created by the spread tissue. Medical procedures using spreading instruments may include gynecological exams, thoracic surgeries, gastrointestinal surgeries, oral surgeries, etc.

During the procedures, spreading of the body tissues requires force for both the initial spreading and to maintain the tissue in spread-apart position. A consistent force often must be applied to the tissue during procedures to prevent collapse or size reduction of the orifice, cavity, or incision. The spreading also is often completed by gloved medical personnel, complicating their use of the spreading instruments.

### SUMMARY OF INVENTION

The present invention provides a medical instrument for spreading apart body tissues that allows for a uniform adjustment of the spacing between the body tissues, and is easily disassemblable and cleanable, such as for sterilization. Exemplary body tissues include human body tissue, such as of a body orifice or incision made in the body.

An exemplary medical device provided by the invention includes two fingers, which can be referred to as blades or paddles or other elongated structures for engaging the body tissue. The fingers extend in a common direction from a common base structure, also referred to as a main body portion. Part of the base structure supports a large nut, through which a threaded first extension coupled to a first one of the fingers is received. This threaded first extension, also referred to as an arm, is typically at an angle relative to the first finger, typically about ninety degrees. Turning the nut may be relatively easy to do while wearing gloves, specifically surgical gloves, to cause the first finger to move toward or away from the other, second, finger without pivoting. In the case that the fingers are parallel, turning the nut can spread the fingers apart while maintaining their parallel relationship.

The second finger is hingedly mounted to the base structure via another extension coupled to the second finger. This second extension, also referred to as an arm, is typically at an angle relative to the second finger, typically about ninety degrees. An angle between the base structure and the second finger, and between the first finger and the second finger, can be adjusted by turning a large bolt, which also may be relatively easily accomplished while wearing gloves. The bolt is secured to the second finger through a passage in the second extension. The bolt engages the base structure so that the angle can change as the large bolt is rotated.

More particularly, the medical instrument provided by the invention includes a main body portion and upper and lower arms coupled to the main body portion that are adjustable relative to the body portion. Fingers, such as blades, extend from the arms for engaging and spreading the tissues. A translating mechanism adjusts to translate at least one of the arms relative to the main body portion to spread apart the tissues, while an adjustment mechanism is adjustable to adjust an angle between the fingers, also to spread apart the tissues. With the fingers disposed close together, the fingers can be inserted into a body orifice or incision.

According to one aspect, a medical instrument for spreading tissue includes a main body portion and upper and lower arms coupled to the main body portion. One of the arms is a translatable arm that translates relative to the main body portion. Upper and lower fingers are coupled to the upper and lower arms respectively for engaging and spreading tissue upon movement of one of the fingers. A translating mechanism adjusts to translate the translatable arm along the main body portion between a primary position adjacent the other of the arms and a secondary position spaced from the primary position. The translatable arm and the main body portion are maintained in uniform and secure engagement in the primary and secondary positions, as well as during translation of translatable arm between the primary and secondary positions via adjustment of the translating mechanism. An adjustment member adjusts an angle between the fingers. The adjustment member engages a lever portion extending from one of the arms and adjusts to move the lever portion towards and away from the main body portion to adjust the angle between the fingers.

The lever portion and one of the fingers may be substantially pivotable about the same axis.

The translating mechanism may include a nut and screw mechanism that is adjustable to translate the translatable arm relative to the main body portion.

The nut may have a diameter greater than one inch.

The translating mechanism may include threads disposed about an outwardly facing surface of the upper arm, and the threads may have Acme thread form.

The adjustment member may be an adjustment bolt that engages the lever portion and the main body portion.

The lever portion may include a slot that receives the adjustment member.

The translating mechanism may include a nut that is rotatable to cause the translation of the translatable arm relative to the main body portion, and the nut may be rotatably engaged with each of the main body portion and the translatable arm.

The main body portion may include an anti-translation portion that maintains the nut in a fixed position along the main body portion.

The nut may rotate about a longitudinal axis along which the translatable arm translates.

The lever portion may be disposed at an acute angle relative to the lower finger.

The main body portion and the lower arm may form an aperture therebetween to provide access between the fingers.

According to another aspect, a medical instrument for spreading tissue includes a main body portion having a longitudinal axis extending therethrough and an upper arm and a lower arm coupled to the main body portion. One of the arms is a translatable arm that is translatable along the longitudinal axis between a primary position adjacent the other of the arms and a secondary position spaced from the primary position. Upper and lower fingers extend respectively from the upper and lower arms for engaging and spreading tissue. One of the fingers pivots relative to the longitudinal axis to spread tissue. A rotatable member is rotatable about the longitudinal axis to effect translation of the translatable arm relative to the other arm to spread tissue.

The translatable arm and the rotatable member may include corresponding threads, and interengagement of the threads may effect translation of the translatable arm along the longitudinal axis.

The rotatable member may be a nut that is threadedly engaged with the translatable arm.

The rotatable member may have a diameter greater than one inch.

The rotatable member may include threads on an inwardly facing surface, and the threads may have Acme thread form.

The main body portion may include an anti-translation portion that maintains the rotatable member in a fixed position along the longitudinal axis relative to the main body portion.

The main body portion and the lower arm may form an aperture therebetween to provide access between the fingers.

According to yet another aspect, a medical instrument for spreading tissue includes a main body portion and an upper arm slideably mounted to the main body portion. The upper arm has outwardly facing threads and an upper finger extending from the upper arm for spreading tissue. A rotatable member is disposed about the main body portion and the upper arm. The rotatable member has inwardly facing threads that engage the outwardly facing threads of the upper arm. A lower arm is pivotably mounted to the main body portion. The lower arm has a lower finger extending from the lower arm for spreading tissue and a lever portion extending from the lower arm in a direction substantially opposite the lower finger. An adjustment member is threadedly mounted to the main body portion and rotatably received by the lever portion. Rotation of the rotatable member effects translation of the upper finger relative to the lower finger. Rotation of the adjustment member effects pivoting of the lower finger relative to the upper finger.

The foregoing and other features of the invention are hereinafter described in greater detail with reference to the accompanying drawings.

### BRIEF DESCRIPTION OF THE DRAWINGS

FIG. 1 is an isometric view of an exemplary spreading instrument according to the invention, taken from the side.

FIG. 2 is another isometric view of the spreading instrument of FIG. 1, taken from the same side.

FIG. 3 is an isometric view of the spreading instrument of FIG. 1, taken from the rear.

FIG. 4 is yet another isometric view of the spreading instrument of FIG. 1, taken from the rear.

FIG. 5 is a partial side elevation view of a portion of the spreading instrument of FIG. 1, showing the same side shown in FIG. 1.

FIG. 6 is an isometric view of a portion of the spreading instrument of FIG. 1.

FIG. 7 is still another isometric view of the spreading instrument of FIG. 1, taken from the top.

FIG. 8 is an isometric view of another exemplary spreading instrument according to the invention.

FIG. 9 is a side elevation view of the spreading instrument of FIG. 8.

FIG. 10 is another side elevation view of the spreading instrument of FIG. 8, showing a side opposite that shown in FIG. 9.

### DETAILED DESCRIPTION

The principles of the present disclosure have particular application to a medical instrument for easily and efficiently spreading apart tissues, such as human body tissues. Of course, the principles of the invention may be useful in other situations, such as to spread apart portions of inanimate objects or tissues of any kind, such as animal or cadaver tissue, where it is desirable to easily and efficiently spread apart tissue or other material.

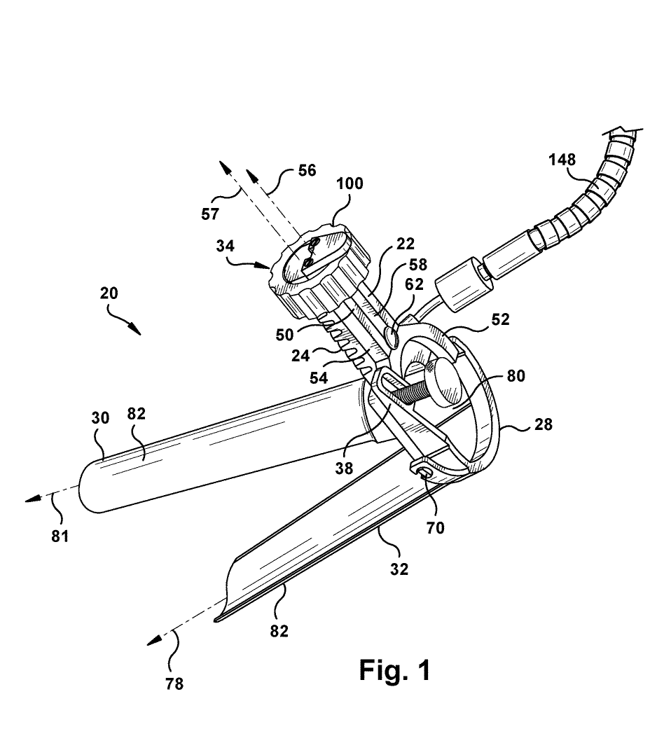

Referring now in detail to FIGS. 1 to 3, a medical instrument 20 is shown for spreading apart tissues, preferably during a medical procedure, such as a cervical exam or an abdominal surgery. The medical instrument 20 is shown in combination with an extension arm 148 for selectively positioning the medical instrument 20 relative to a surface to which the extension arm 148 is attached.

As shown, the medical instrument 20 includes a main body portion 22 which may be held by a user to control the instrument 20. Coupled to the main body portion 22 are upper and lower arms 24 and 28 that control movement of respective upper and lower fingers 30 and 32 extending from the upper and lower arms 24 and 28. The fingers 30 and 32 are provided for engaging and spreading apart tissues. A translating mechanism 34 and an angular adjustment mechanism 38 are provided for controlling precise and incremental adjustment of the arms 24 and 28, and thus of the fingers 30 and 32, relative to each other and relative to the main body portion 22.

Each of the main body portion 22, arms 24 and 28, fingers 30 and 32, translating mechanism 34, and angular adjustment mechanism 38 generally may be comprised of a material that is suitable for autoclave sterilization. Suitable materials may include, but are not limited to, stainless steel, titanium, etc.

The main body portion 22 includes an upper arm engagement portion 50 and a lower arm engagement portion 52. The upper arm engagement portion 50 linearly extends from the lower arm engagement portion 52 along a main longitudinal axis 56 and provides a translation surface 54 against which the upper arm 24 translates. The upper arm 24 translates along a translation longitudinal axis 57 that is disposed substantially parallel to the main longitudinal axis 56.

A slot 58 extends through the upper arm engagement portion 50 for receiving a fastener 62. The fastener 62 may be any suitable device, such as a screw, bolt, etc. The depicted fastener 62 is coupled to the upper arm 24 and is adjustable relative to each of the upper arm 24 and the upper arm engagement portion 50 to maintain the upper arm 24 in slideable engagement with the translation surface 54. During translation of the upper arm 24 relative to the main body portion 22, the fastener 62 slides relative to the upper arm engagement portion 50, such as within the slot 58.

The illustrated fastener 62 may be tightened, such as adjusted relative to the upper arm 24, to fix the translatable position of the upper arm 24 relative to the main body portion 22. In some embodiments, the fastener 62 may include a handle (not shown) that may be easily turned by a user of the instrument 20. The depicted fastener 62 is a threaded fastener, such as a screw or bolt, which is threadedly coupled to the upper arm 24. The fastener 62 may be removed from the upper arm 24, such as by using a tool, for example, a screwdriver, to disassemble the instrument 20 for cleaning.

In other embodiments the fastener 62 may be omitted and one of the upper arm 24 and the upper arm engagement portion 50 may include a longitudinally extending T-slot or L-slot. The other of the upper arm 24 and the upper arm engagement portion 50 may include a key corresponding to the slot and slideable within the slot. The slot and key may enable laterally-fixed, transversely-fixed, longitudinal translation of the upper arm 24 relative to the main body portion 22, while maintaining placement of the upper arm 24 and upper arm engagement portion 50 adjacent one another. As used herein, the lateral and transverse directions are directions disposed orthogonal to the longitudinal direction and orthogonal to one another. The longitudinal direction is parallel the longitudinal axes 56 and 57.

Disposed opposite the upper arm engagement portion 50 is the lower arm engagement portion 52, which is configured to couple to the lower arm 28. As shown, the main body portion 22 includes pinions 70 extending from the lower arm engagement portion 52. The pinions 70 extend outwardly along attachment portions 75 to respective pinion heads 76, disposed at the distal ends of the pinions 70. The pinions 70 extend away from the lower engagement portion 52 along a single pinion axis 77, though in other embodiments, the pinions may extend along different axes.

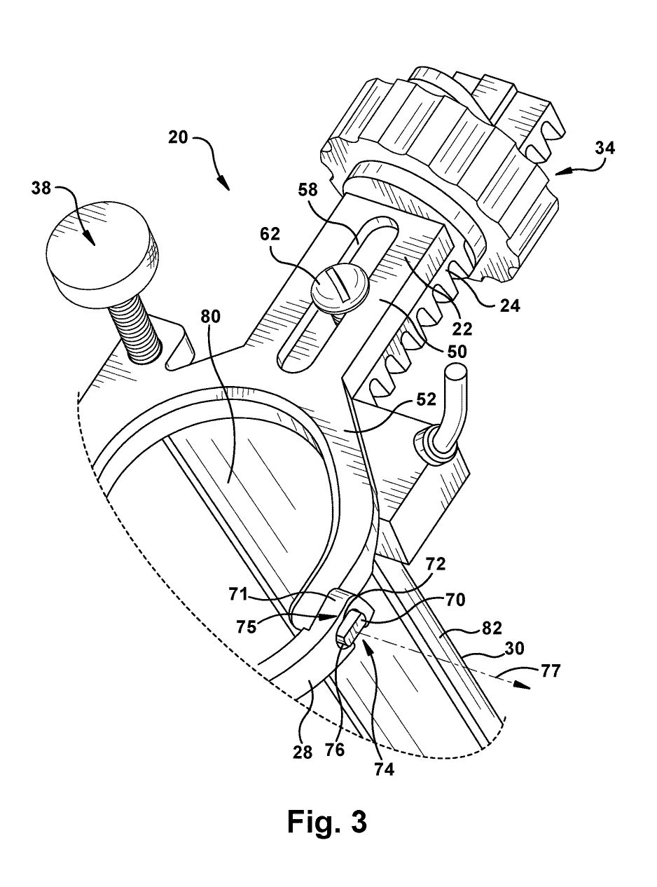

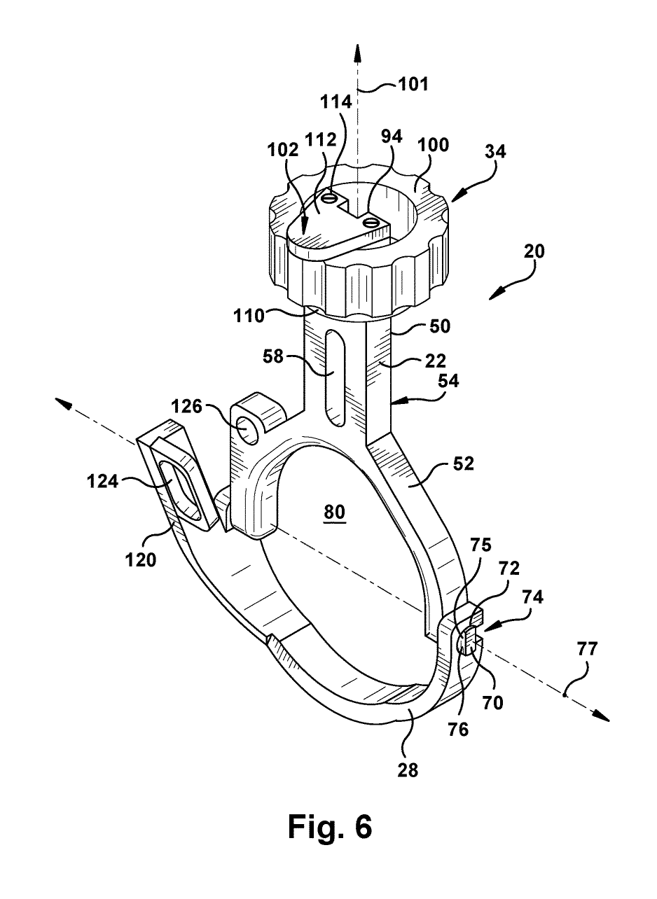

The attachment portions 75 of the pinions 70 are received by corresponding slots 72 of slotted portions 71 of the lower arm 28. As shown best in FIG. 3 (and also in FIG. 6), the slots 72 include slot breaks 74 that allow the attachment portion 75 of each pinion 70 to be moved into the respective slot 72. The slot breaks 74 extend through the respective slotted portions 71 of the lower arm 28 in a radially outward direction from a center of the slots 72.

The slotted portions 71 are engaged between the engagement portion 52 and the pinion heads 76, and the slots 72 extend at least partially about the attachment portions 75. In this way, the slotted portions 71 are retained about the pinions 70 for rotation of the lower arm 28 relative to the main body portion 22.

In some embodiments each attachment portion 75 of each pinion 70 may have a width dimension and a height dimension, and each of these dimensions may extend orthogonal to the length of the respective attachment portion 75 that extends along the pinion axis 77. The width and height dimensions of the pinions 70 may not be equal, and thus one of the width and height may be greater than the other of the width and the height.

In these embodiments, a width of the respective slot break 74 extends substantially orthogonal to the respective pinion axis 77. The width of the slot break 74 is sized large enough to receive only one of the width and height of the respective pinion 70. Thus the lower arm 28 may be pivotably coupled to the lower arm engagement portion 52 when the width of the slot break 74 is rotated relative to the main body portion 22 such that it may receive the narrower of the width and height dimension of the pinion 70. Once the pinion 70 is received in the slot 72, the lower arm 28 may be rotated about the pinion axes. The rotation aligns the width of the slot break 74 with the larger of the width and height dimensions of the respective pinion 70, which is larger than the width of the slot break 74. Thus removal of the pinion 70 from the slot 72 is prevented until the lower arm 28 is again rotated a sufficient amount.

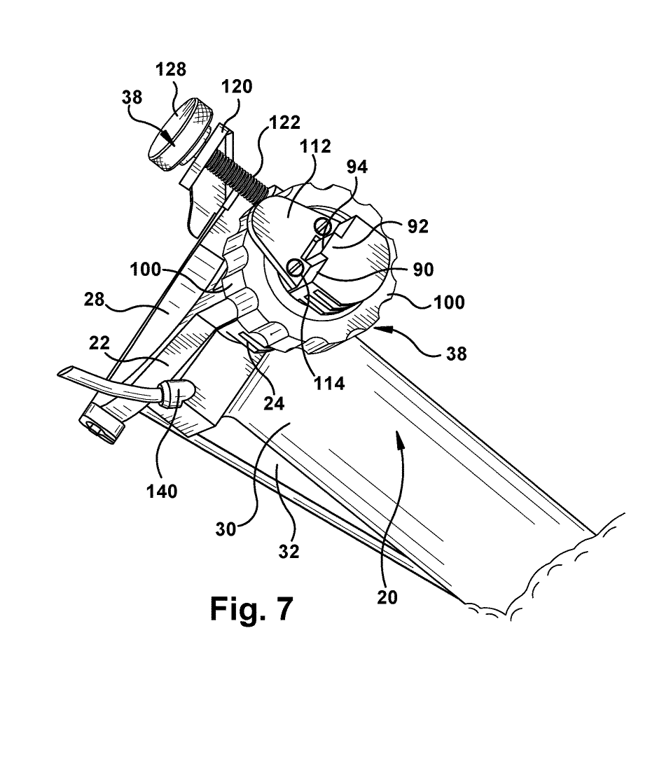

For example, the slot break 74 is aligned to receive the pinion 70 when a lower finger longitudinal axis 78 of the lower finger 32, along which the lower finger 32 extends outwardly from the lower arm 28, is aligned substantially parallel to the main longitudinal axis 56. The slot break 74 is aligned to prevent removal of the pinion 70 from the slot 72 when the longitudinal axis of the lower finger 28 is rotated out of a substantially parallel relationship with the main longitudinal axis 56. This coupling of the lower arm 28 and lower arm engagement portion 52 provides for easy disassembly of the lower arm 28 from the main body portion 22 for cleaning but makes it unlikely that these parts may inadvertently separate during use. (See also FIG. 7.)

While two slots 72 and two pinions 70 are depicted, any suitable number of slots and pinions may be used. In other embodiments, the main body portion 22 may include the slots 72 and the lower arm 28 may include the corresponding pinions 70.

The two slot and pinion sets are oppositely disposed at opposite sides of an aperture 80 defined at least in part by the lower engagement portion 52 of the main body portion 22 and the lower arm 28. In the illustrated embodiment, each of the lower engagement portion 52 and the lower arm 28 have curved shapes, such as bowed shapes, defining the aperture 80.

As shown, the aperture 80 lies between the fingers 30 and 32, such that a user may use the aperture 80 for viewing between the fingers 30 and 32, or for placing additional devices, such as medical devices, through the aperture to access tissue between the tissue being spread by the fingers 30 and 32. Notably, the translating mechanism 34 and the angular adjustment mechanism 38 are spaced from the aperture 80 to provide unimpeded access to the aperture 80.

The upper and lower fingers 30 and 32 are spaced about opposite sides of the aperture and as explained, movement of the fingers 30 and 32 relative to one another is effected by the translating mechanism 34 and the angular adjustment mechanism 38. Similar to the lower finger 32, the upper finger 30 extends outwardly from the upper arm 24 along an upper finger longitudinal axis 81. The upper finger 30 extends outwardly along the upper finger longitudinal axis 81 which is disposed substantially parallel to the main longitudinal axis 56. Both fingers generally extend in a common direction. Prior to adjustment, the respective upper and lower finger longitudinal axes 81 and 78 are disposed substantially parallel to one another and adjacent one another.

The depicted upper and lower fingers 30 and 32 are shown as curved blades which are inwardly curved towards one another. Convex radially outward surfaces 82 of the fingers 30 and 32 are configured to engage the body tissue being spread. One or more of the fingers 30 and 32 may include slots for receiving and maintaining position of other instrumentation used with the medical instrument 20.

In other embodiments, the fingers 30 and 32 may be of any other suitable shape, such as flat blades, rods, tubes, or shapes that extend angularly from one another. In some embodiments, the fingers 30 and 32 may include two or more portions which are translatable relative to one another along the longitudinal finger axes 78 and 81.

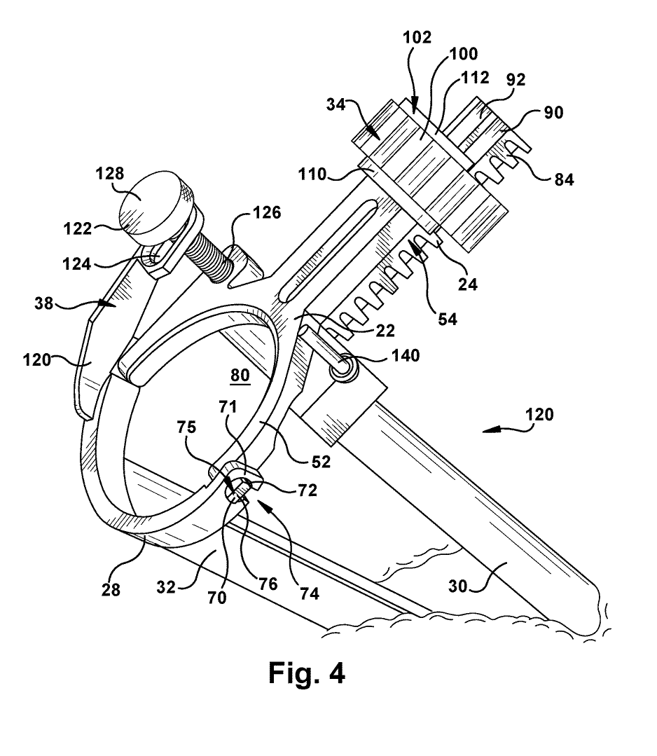

Turning now to FIGS. 4 to 7, the upper arm 24, which is coupled to the upper finger 30, includes a longitudinal engagement portion 84 that is configured to translate along the translation surface 54 of the upper engagement portion 50 of the main body portion 22. An inwardly facing surface 90 of the upper arm 24 includes a key 92 extending along a longitudinal length of the longitudinal engagement portion 84. The key 92 is received by a corresponding slot 94 disposed in the translation surface 54 of the upper engagement portion 50.

The engagement of the key 92 and slot 94 maintains lateral positioning of the upper arm 24 relative to the main body portion 22 during translation of the upper arm 24 relative to the main body portion 22. The engagement of the key 92 and the slot 94 also maintains lateral positioning of the upper finger 30 relative to the lower finger 32 during translation of the upper arm 24.

To translate the translatable upper arm 24, the medical instrument 20 includes the translating mechanism 34, which enables the upper finger 30 to move without pivoting or rotating relative to the lower finger 32 as the upper finger 30 moves. The translatable upper arm 24 is movable between a primary position adjacent the lower arm 28 and a secondary position spaced from the primary position where the upper arm 24 is further spaced from the lower arm 28.

The translating mechanism 34 includes a rotatable member 100 and an anti-translation portion 102 that cooperate to enable incremental translation of the upper arm 24. In this way, the translating mechanism 34 maintains uniform and secure engagement of the upper arm 24 and the main body portion 22 at each of the primary and secondary positions and during the translation of the upper arm 24 and upper finger 30. As used herein, uniform and secure engagement means that spacing between the engagement surface 54 and the upper arm 24 is uniform and does not change at each of the primary and secondary positions and during the translation of the upper arm 24. Thus, the depicted translation mechanism 34 is not necessarily loosened to enable translation of the upper arm 24 and then again tightened once the translation is completed.

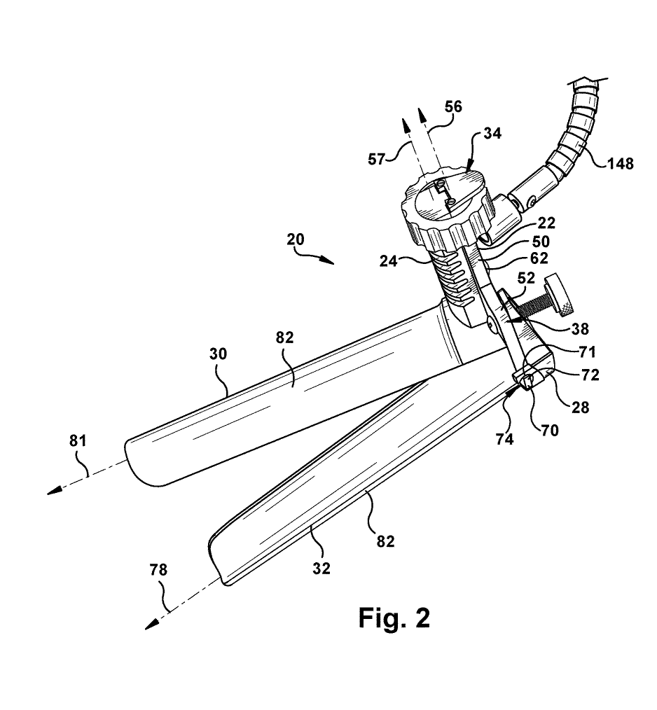

The uniform and secure engagement is enabled via the rotatable member 100 that rotatably engages each of the upper arm engagement portion 50 and the upper arm 24. The rotatable member 100 is disposed about each of the upper arm engagement portion 50 and the upper arm 24 and rotates about a longitudinal axis 101 (FIG. 6), disposed parallel to the longitudinal axes 56 and 57 (FIGS. 1 and 2).

Rotation of the rotatable member 100 effects the translation of the upper arm 24 relative to the main body portion 22. As shown, the rotatable member 100 is a nut that is threadedly engaged to the upper arm 24. The nut 100 includes threads on an inwardly facing surface, and the upper arm 24 includes corresponding threads on an outwardly facing surface. The threads of the nut 100 and the threads on the upper arm 24 cooperate to enable translation of the upper arm 24 relative to the main body portion 22 and relative to the lower arm 28 as the nut 100 is rotated. Thus the translating mechanism 34 includes a nut and screw mechanism that is adjustable to translate the translatable upper arm 24 relative to the main body portion 22.

The threaded translation mechanism 34 provides the advantage of not relying on a friction fit between components to maintain positioning of the upper finger 30 relative to the lower finger 32. This is particularly the case when the medical instrument 20 applies a spreading force to tissue that applies a substantial return force against the fingers 30 and 32. In such case, the corresponding threads of the rotatable member 100 and the upper arm 24 maintain the upper arm 24 and main body portion 22 in a temporarily-fixed engagement until the rotatable member 100 is rotated again to effect translation of the upper arm 24.

The threads of the rotatable member 100 and the upper arm 24 preferably have Acme thread form, although any other suitable thread form may be used. The Acme thread form may be used due to the high strength, ease of manufacture, and ease of rotation of the rotatable member/nut 100.

The nut 100 is sized and shaped to be easily handled by an individual wearing hand coverings, such as surgical gloves. An outwardly facing surface of the nut 100 includes ridges for enabling traction of a gloved hand against the nut 100. The nut 100 also is large enough that it may be easily gripped by hand. For example, the nut 100 may have an outer diameter greater than 1 inch, and more preferably greater than 2 inches.

The nut/rotatable member 100 is maintained in a fixed position along the main body portion 22, and along the main longitudinal axis 56 (FIGS. 1 and 2), via the anti-translation portion 102. The anti-translation portion 102 includes a lower member 110 and an upper member 112. The illustrated lower member 110 is shown as coupled to a top portion of the upper arm engagement portion 50 and is disposed below the rotatable member 100. The illustrated upper member 112 is shown as coupled to the top surface of the upper arm engagement portion 50, and thus the rotatable member 100 is disposed between upper member 112 and the lower member 110.

The upper member 112 is coupled to the top surface of the main body portion 22 by fasteners 114, such as bolts, screws, etc. In this way, removal of at least the fastener 62 and the fasteners 114 enables disassembly of the main body portion 22 from the upper arm 24/upper finger 30.

In some embodiments, a stop member (not shown) may be coupled to a top surface of the upper arm 24. During downward translation of the upper arm 24 in a direction towards the lower arm 28, the stop member will be caused to engage at least one of the rotatable member 100 or the upper member 112, for example. In this way, the upper arm 24 may not be continually translated through the rotatable member 100 to a point where it disengages from the rotatable member 100. The stop member may be coupled to the top surface of the upper arm 24 by any suitable means, such as welding, fasteners, etc., though fasteners are preferable to facilitate disassembly.

In addition to the translation mechanism 34 for effecting translation of the upper and lower fingers 30 and 32 relative to one another, the instrument 20 also includes the angular adjustment mechanism 38 that is configured to adjust an angle between the fingers 30 and 32. The angular adjustment mechanism 38 includes a lever portion 120 coupled to the lower arm 28. An adjustment member 122, such as an adjustment bolt, engages each of the lever portion 120 and the lower arm engagement portion 52 of the main body portion 22. Adjustment of the adjustment member 122, such as through rotation as further described below, effects pivoting of the lever portion 120, such that the lever portion 120 moves toward and away from the main body portion 22 to adjust an angle between the fingers 30 and 32.

The lever portion 120 extends from the lower arm 28 in a direction substantially opposite the lower finger 28, and more preferably at an acute angle relative to the lower finger 28. A slot 124, or other suitable opening, in the lever portion 120 is sized to receive the adjustment member 122. The slot 124 also is sized to allow pivoting of the lever portion 120 and lower arm 28 relative to the main body portion 22.

The adjustment member 122 includes threads on an outwardly facing surface that threadedly engage threads of a threaded portion 126 (FIGS. 6, 14), such as a threaded depression, of the lower arm engagement portion 52 of the main body portion 22. Accordingly, rotation of the adjustment member 122 that is received through the slot 124 causes the movement of the lever portion 120 relative to the main body portion 22. Additionally, due to the engagement of the pinions 70 and slots 72, rotation of the adjustment member 122 causes the lever portion 120 and the lower finger 32 to pivot about the same axis, which is the pinion axis.

The adjustment member 122 includes a grip portion 128 which is configured, such as sized, to enable manipulation of the adjustment member 122 by gloved personnel. The grip portion 128 may include raised ridges on an outwardly facing surface to provide aid in traction between the trip portion 128 and a hand or tool.

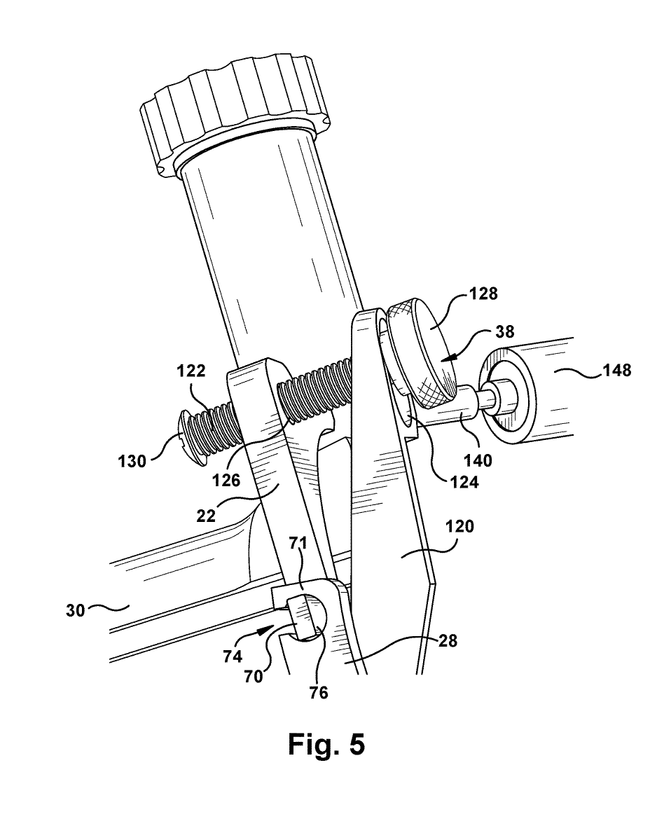

Further, the adjustment member 122 may include a stop member 130 (FIG. 5), such as a nut, disposed at an end opposite the grip portion 128. The stop member 130 may be configured to engage the main body portion 22 such that during adjustment of the adjustment member 122, the adjustment member 122 may not be fully disengaged from the main body portion 22.

In other embodiments, the lever portion 120 may be coupled to the main body portion 22 and the adjustment fastener 122 may be threadedly received by the lower arm 28. In still other embodiments, the lower arm 28 may be configured to translate relative to the upper arm 24 and/or the upper arm 24/upper finger 30 may be configured to pivot relative to the lower arm 28/lower finger 32. In some embodiments, the upper arm 24 may be configured to both translate relative to the lower arm 28 and to pivot relative to the lower arm 28. In some embodiments both of the lower and upper arms 24 and 28 may be configured to translate relative to one another and/or to pivot relative to one another.

The medical instrument 20 also may include at least one auxiliary attachment member. As shown, an auxiliary attachment member 140 is coupled to the upper arm 24 and is provided for coupling to another device, such as a light, camera, or the extension arm 148. As shown, the extension arm 148 is a flexible arm that may be loosened for adjustment in each of the six degrees of freedom. The extension arm 148 is removably coupled to the auxiliary attachment member 140 such that the instrument 20 may be used in combination with or separately from the extension arm 148.

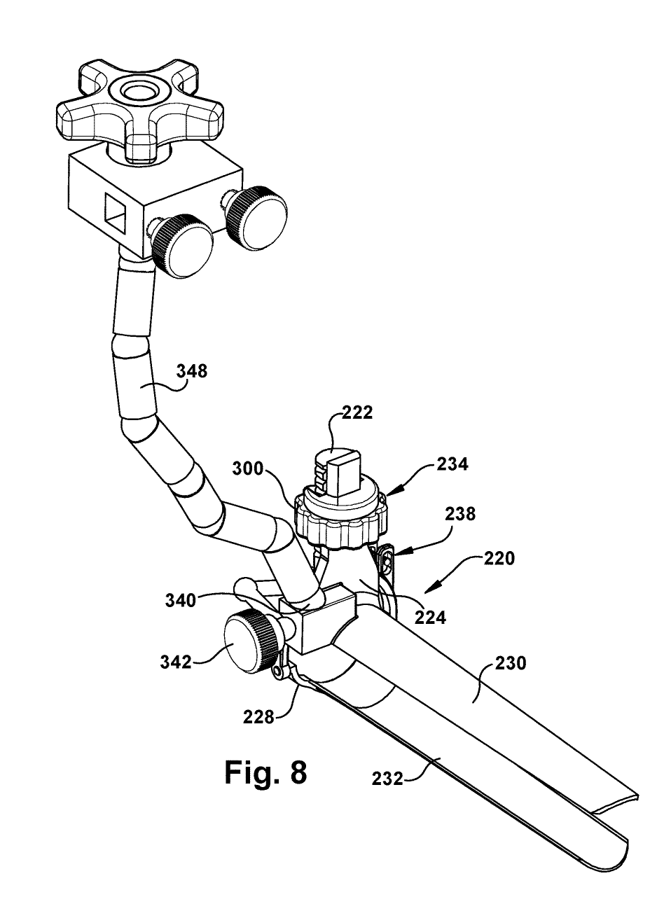

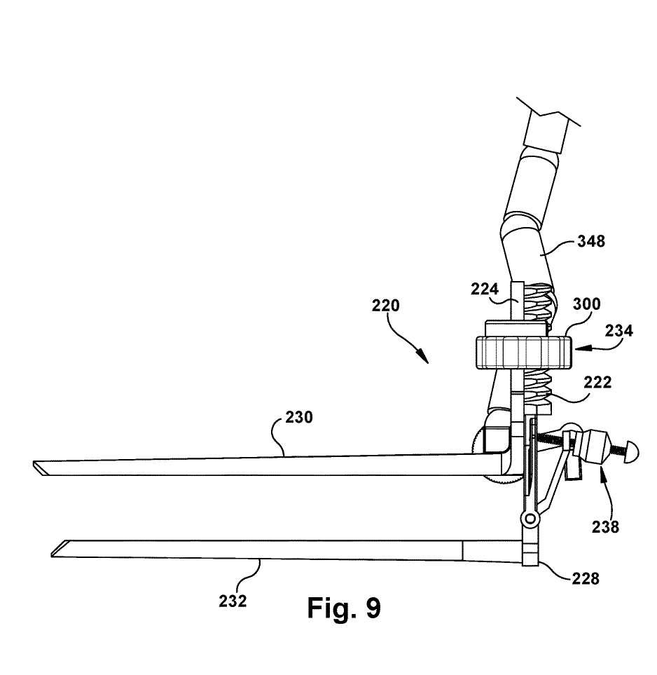

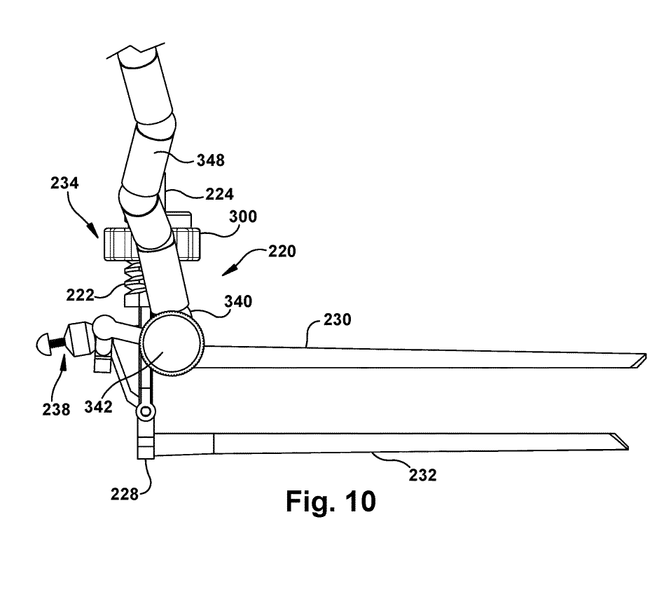

Turning now to FIGS. 8 to 10, another exemplary embodiment of a medical instrument according to the invention is shown at 220. The instrument 220 is substantially the same as the above-referenced instrument 20, and consequently the same reference numerals, indexed by 200, are used to denote structures corresponding to similar structures in the instrument 220. In addition, the foregoing description of the instrument 20 is equally applicable to the instrument 220 except as noted below. Moreover, aspects of the instruments 20 and 220 may be substituted for one another or used in conjunction with one another where applicable.

As illustrated, the instrument 220 includes a main body portion 222, an upper arm 224, a lower arm 228, an upper finger 230, a lower finger 232, a translation mechanism 234, and an adjustment mechanism 238. Unlike the instrument 20, the main body portion 222 includes threads on an outwardly facing surface for engaging corresponding threads on an inwardly facing surface of a rotatable member 300. The upper arm 224 is translatably-fixed relative to the rotatable member 300 such that it does not translate separate from the rotatable member 300.

Further, the instrument 220 includes an auxiliary attachment member 340 that is coupled to the upper arm 224 and is provided for coupling to another device, such as a light, camera, or an extension arm 348. The auxiliary attachment member 340 may include an adjustment handle 342 that may be loosened to adjust the positioning of the other instrument 220, and then tightened to maintain that positioning. As shown, the extension arm 348 is a flexible arm that may be loosened for adjustment in each of the six degrees of freedom. The extension arm 348 is removably coupled to the auxiliary attachment member 340 such that the instrument 220 may be used in combination with or separately from the extension arm 348.

In summary, the invention provides a spreading instrument 20/220 for spreading tissue. The instrument 20/220 includes a main body portion 22/222 and upper and lower arms 24/224 and 28/228 coupled to the main body portion 22/222. One of the arms 24/224 or 28/228 is a translatable arm that translates relative to the main body portion 22/222. Upper and lower fingers 30/230 and 32/232 are coupled to the upper and lower arms 24/224 and 28/228 respectively for engaging and spreading the tissue upon movement of one of the fingers 30/230 or 32/232. A translating mechanism 34 adjusts to translate the translatable arm 24/224 or 28/228 along the main body portion 22/222 between a primary position adjacent the other of the arms 24/224 or 28/228 and a secondary position spaced from the primary position. An adjustment member 122 engages a lever portion 120 extending from one of the arms 24 or 28 and causes movement of the lever portion 120 towards and away from the main body portion 22 to adjust an angle between the fingers 30 and 32.

Although the invention has been shown and described with respect to a certain embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

1. A medical instrument for spreading tissue, the medical instrument comprising:
a main body portion;
upper and lower arms coupled to the main body portion, wherein one of the arms is a translatable arm that translates relative to the main body portion;
upper and lower fingers coupled to the upper and lower arms respectively for engaging and spreading tissue upon movement of one of the fingers;
a translating mechanism that adjusts to translate the translatable arm along the main body portion between a primary position adjacent the other of the arms and a secondary position spaced from the primary position, the translatable arm and the main body portion being maintained in uniform and secure engagement in the primary and secondary positions, as well as during translation of translatable arm between the primary and secondary positions via adjustment of the translating mechanism;
an adjustment member that adjusts an angle between the fingers, wherein the adjustment member engages a lever portion extending from one of the arms and adjusts to move the lever portion towards and away from the main body portion to adjust the angle between the fingers.

2. The medical instrument of claim 1, wherein the lever portion and one of the fingers are substantially pivotable about the same axis.

3. The medical instrument of claim 1, wherein the translating mechanism includes a nut and screw mechanism that is adjustable to translate the translatable arm relative to the main body portion.

4. The medical instrument of claim 3, wherein the nut has a diameter greater than one inch.

5. The medical instrument of claim 1, wherein the translating mechanism includes threads disposed about an outwardly facing surface of the upper arm, and wherein the threads have Acme thread form.

6. The medical instrument of claim 1, wherein the adjustment member is an adjustment bolt that engages the lever portion and the main body portion.

7. The medical instrument of claim 1, wherein the lever portion includes a slot that receives the adjustment member.

8. The medical instrument of claim 1, wherein the translating mechanism includes a nut that is rotatable to cause the translation of the translatable arm relative to the main body portion, and wherein the nut is rotatably engaged with each of the main body portion and the translatable arm.

9. The medical instrument of claim 8, wherein the main body portion includes an anti-translation portion that maintains the nut in a fixed position along the main body portion.

10. The medical instrument of claim 8, wherein the nut rotates about a longitudinal axis along which the translatable arm translates.

11. The medical instrument of claim 8, wherein the lever portion is disposed at an acute angle relative to the lower finger.

12. The medical instrument of claim 1, wherein the main body portion and the lower arm forms an aperture therebetween to provide access between the fingers.

13. A medical instrument for spreading tissue, the medical instrument comprising:
a main body portion having a longitudinal axis extending therethrough;
an upper arm and a lower arm coupled to the main body portion, wherein one of the arms is a translatable arm that is translatable along the longitudinal axis between a primary position adjacent the other of the arms and a secondary position spaced from the primary position;
upper and lower fingers extending respectively from the upper and lower arms for engaging and spreading tissue, wherein one of the fingers pivots relative to the longitudinal axis to spread tissue;
a rotatable member rotatable about the longitudinal axis to effect translation of the translatable arm relative to the other arm to spread tissue.

14. The medical instrument of claim 13, wherein the translatable arm and the rotatable member include corresponding threads, and wherein interengagement of the threads effects translation of the translatable arm along the longitudinal axis.

15. The medical instrument of claim 13, wherein the rotatable member is a nut that is threadedly engaged with the translatable arm.

16. The medical instrument of claim 13, wherein the rotatable member has a diameter greater than one inch.

17. The medical instrument of claim 13, wherein the rotatable member includes threads on an inwardly facing surface, and wherein the threads have Acme thread form.

18. The medical instrument of claim 13, wherein the main body portion includes an anti-translation portion that maintains the rotatable member in a fixed position along the longitudinal axis relative to the main body portion.

19. The medical instrument of claim 13, wherein the main body portion and the lower arm forms an aperture therebetween to provide access between the fingers.

20. A medical instrument for spreading tissue, the medical instrument comprising:
a main body portion;
an upper arm slideably mounted to the main body portion, the upper arm having outwardly facing threads, and the upper arm having an upper finger extending from the upper arm for spreading tissue;
a rotatable member disposed about the main body portion and the upper arm, the rotatable member having inwardly facing threads that engage the outwardly facing threads of the upper arm;
a lower arm pivotably mounted to the main body portion, the lower arm having a lower finger extending from the lower arm for spreading tissue, and the lower arm having a lever portion extending from the lower arm in a direction substantially opposite the lower finger;
an adjustment member threadedly mounted to the main body portion and rotatably received by the lever portion;
wherein rotation of the rotatable member effects translation of the upper finger relative to the lower finger, and wherein rotation of the adjustment member effects pivoting of the lower finger relative to the upper finger.
